(19) United States
(12) Patent Application Publication
(10) Pub. No.: US 2025/0169974 A1
(43) Pub. Date: May 29, 2025

(54) DEVICES, SYSTEMS AND METHODS FOR ACCESS AND DELIVERY TO CHALLENGING ANATOMY

(71) Applicant: BOSTON SCIENTIFIC SCIMED, INC., Maple Grove, MN (US)

(72) Inventors: Peter L. Dayton, Brookline, MA (US); Thomas DeSimio, Knoxville, TN (US); Mark Phillip Olson, Saint Anthony, MN (US); Aaron Abbott, Columbia Heights, MN (US); Natalie Byrom, Boston, MA (US)

(73) Assignee: BOSTON SCIENTIFIC SCIMED, INC., Maple Grove, MN (US)

(21) Appl. No.: 18/960,832

(22) Filed: Nov. 26, 2024

Related U.S. Application Data
(60) Provisional application No. 63/603,396, filed on Nov. 28, 2023.

(51) Int. Cl. A61F 2/966; A61F 2/95

(52) U.S. Cl. CPC A61F 2/966; A61F 2/9517

(57) ABSTRACT

Delivery devices, systems and procedures are disclosed, for delivering an implant, such as a stent, including an outer sheath, an inner member slideably disposed within the outer sheath, a handle housing and a handle actuator slideably disposed within the handle housing, a pusher member with inner member attached that is pushable distally relative to the handle actuator to extend the inner member relative to the outer sheath, and a puller member with outer sheath attached pullable proximally relative to the handle actuator to retract the outer sheath relative to the outer sheath.

### CROSS REFERENCE TO RELATED APPLICATIONS

The present application claims the benefit of and priority to U.S. Provisional Patent Application Ser. No. 63/603,396, filed on Nov. 28, 2023, the disclosure of which is incorporated herein by reference.

### TECHNICAL FIELD

The disclosure pertains to medical delivery devices, systems and methods, and more particularly to delivery devices for delivering implants such as stents into challenging anatomy such as confined or limited spaces in the body.

### BACKGROUND

A wide variety of medical devices have been developed for medical use including, for example, medical devices utilized to access anatomy and to deliver and deploy medical implants, such as stents, within and into a patient's body. Stent placement can be necessary to connect or appose body lumens, drain from one body lumen to another, close strictures, repair and provide support to structures, etc. For example, current devices and methods of stent deployment generally involve endoscopic delivery of a stent to a location within the body. Of the known delivery devices and methods for deploying stents, each has certain advantages and disadvantages. It may be desirable to provide medical devices that allow more precise or controlled access and placement of stents during endoscopic procedures, especially procedures involving challenging anatomy, such as small spaces, narrow lumens, requiring a complicated angle of approach, into lumens that are typically not expanded when empty, etc. Many current devices depend solely on pulling motions to deploy a stent-such as devices where you can only retract an external sheath from about an internal shaft to expose a self-expanding stent—which may pose problems when anatomy requires static placement or would benefit from forward placement. It may be difficult to deploy a stent in certain places when relying on only retraction of a sheath, perhaps leading to incorrect placement angles, deploying too far proximally, and/or deploying within a channel etc.

It is with these considerations in mind that the designs, features and embodiments of the present disclosure may be advantageous.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiments.

In one aspect of the disclosure, a delivery device may comprise a handle housing, a handle actuator, a puller member, a pusher member, an inner member and an outer sheath. The outer sheath has a proximal end, a distal end, and a lumen extending longitudinally therethrough. The inner member may be slideably disposed within the lumen of the outer sheath. The inner member may have a distal tip disposed on a distal end of the inner member. The handle housing has a proximal end, a distal end, and a lumen extending longitudinally therethrough. The handle actuator may be slideably disposed within the lumen of the handle housing. The puller member may be attached to the proximal end of the outer sheath. The puller member and outer sheath may be slideably associated with the handle actuator, and the pusher member may be attached to the proximal end of the inner member. The pusher member and inner member may be slideably associated with the handle actuator and the outer sheath. The outer sheath and inner member may be fixable against sliding with respect to the handle actuator, such that translation of the handle actuator relative to the handle housing may adjust the effective working length of the outer sheath and inner member distal to the handle housing. Translation of the puller member may slide the outer sheath proximally within the handle actuator and from about the inner member, and translation of the pusher member may slide the inner member distally from within the outer sheath.

In embodiments disclosed and otherwise within the scope of the present disclosure, a delivery device may comprise a stopper. The stopper may have a lock. The stopper may be fixed in position relative to the handle actuator and disposed along the device between the puller member and the pusher member. The lock when engaged may be configured to hold the pusher member in place from sliding relative to the stopper. The handle housing may further comprise a lock. The lock when engaged may be configured to hold the handle actuator in place from sliding relative to the handle housing. The handle actuator may further comprise a lock. The lock when engaged may be configured to hold the puller member in place from sliding relative to the handle actuator. The handle actuator may further comprise one or more rails extending proximally and longitudinally along the device. The stopper may be fixed to a proximal end of the handle actuator rails and disposed along a predetermined longitudinal position of the device between the puller member and the pusher member. The stopper may be configured as a stop against both the puller member being translated proximally and the pusher member being translated distally, beyond the predetermined position. The pusher member may further comprise one or more rails extending distally and longitudinally along the device. The pusher member rails may be configured to slide along the handle actuator rails. The puller member may further comprise channels. The channels may be configured to guide the puller member when the puller member is slid proximally along the handle actuator rails. A delivery device may comprise a stent constrained for delivery. The stent may be constrained for delivery within the lumen of the outer sheath between the outer sheath and the inner member. The distal tip of the inner member may extend distally from the distal end of the outer sheath. The inner member may be moved distally by a user pushing on the pusher member, wherein a distal end of the stent may be deployed. The outer sheath may be moved proximally by the user pulling on the puller member, wherein a proximal end of the stent may be deployed. When the stent is fully deployed, it may be shaped and sized such that the distal tip of the inner member may be retracted proximally through the stent. The delivery device may include a distal tip with a conductive area configured to cut through a tissue wall. Electrical energy may be supplied to the distal tip through a plug disposed on the pusher member. Radiofrequency energy may be supplied to the distal tip through an electrical plug on the pusher member. A knob may be fixedly attached to the handle actuator.

In another aspect of the disclosure, a system may comprise a delivery device and a stent. The delivery device may comprise a handle housing, a handle actuator, a puller member, a pusher member, an inner member and an outer sheath. The outer sheath has a proximal end a distal end, and a lumen extending longitudinally therethrough. The inner member may be slideably disposed within the lumen of the outer sheath. The inner member may have a distal tip disposed on a distal end of the inner member. The handle housing has a proximal end, a distal end, and a lumen extending longitudinally therethrough. The handle actuator may be slideably disposed within the lumen of the handle housing. The puller member may be attached to the proximal end of the outer sheath. The puller member and outer sheath may be slideably associated with the handle actuator, and the pusher member may be attached to the proximal end of the inner member. The pusher member and inner member may be slideably associated with the handle actuator and the outer sheath. The outer sheath and inner member may be fixable against sliding with respect to the handle actuator, such that translation of the handle actuator relative to the handle housing may adjust the effective working length of the outer sheath and inner member distal to the handle housing. Translation of the puller member may slide the outer sheath proximally within the handle actuator and from about the inner member. Translation of the pusher member may slide the inner member distally from within the outer sheath. The stent may be self-expanding and may be constrained on the inner member between the inner member and the outer sheath and may be deployable therefrom by first pushing the inner member with the pusher member relative to the outer sheath and then pulling the outer sheath with the pulling member relative to the inner member.

In embodiments disclosed and otherwise within the scope of the present disclosure, when the stent is fully deployed, it may be shaped and sized such that the distal tip of the inner member may be retracted proximally through the stent. The handle actuator may further comprise one or more rails extending proximally and longitudinally along the device. The handle housing may further comprise a stopper. The stopper may be fixed to a proximal end of the handle actuator rails and disposed along a predetermined longitudinal position of the device between the puller member and the pusher member. The stopper may be configured as a stop against both the puller member being translated proximally and the pusher member being translated distally, beyond the predetermined position. The pusher member may further comprise one or more rails extending distally and longitudinally along the device. The pusher member rails may be configured to slide along the handle actuator rails. The puller member may further comprise channels. The channels may be configured to guide the puller member when the puller member is slid proximally along the handle actuator rails. The distal tip of the inner member may extend distally from the distal end of the outer sheath. The delivery device may include the distal tip with a conductive area configured to cut through a tissue wall. Electrical energy may be supplied to the distal tip through a plug disposed on the pusher member. Radiofrequency energy may be supplied to the distal tip through an electrical plug on the pusher member. A knob may be fixedly attached to the handle actuator.

In a further aspect of the disclosure, a method may comprise positioning a delivery device in a body of a patient proximate adjacent first and second tissue walls within the body. The device may be carrying a stent. The delivery device may comprise a handle body, a handle housing, a handle actuator, a puller member, a pusher member, an inner member and an outer sheath. The inner member may be slidable within a lumen of the outer sheath. The handle body may comprise the handle actuator slidable through a lumen of the handle body. The puller member may be attached to the outer sheath and may be slidable relative to the handle actuator. The pusher member may be attached to the inner member and may be slidable relative to the handle actuator. The method may comprise actuating the handle actuator, causing distal translation of the outer sheath and inner member through the first tissue wall and second tissue wall; pushing the pusher member and inner member distally relative to the handle body and outer sheath, causing deployment of the inner member and a distal end of the stent from within the outer sheath; translating the inner member and outer sheath together proximally relative to the handle body to engage the deployed distal end of the stent against the second tissue wall; and pulling the puller member and outer sheath proximally relative to the handle body and inner member, causing deployment of the proximal end of the stent against the first tissue wall and securing the first and second tissue walls between the distal and proximal stent ends.

In the embodiments disclosed or otherwise within the scope of the present disclosure, the device of a method may further comprise a stopper having distal and proximal surfaces. The stopper may be fixed in position relative to the handle actuator between the pusher member and puller member. Distal movement of the pusher member and inner member during the pushing step may be limited by the pusher member butting up against the proximal surface of the stopper. Proximal movement of the pulling member and outer sheath during the pulling step may be limited by the puller member butting up against the distal surface of the stopper. The handle actuator may further comprise one or more rails extending proximally and longitudinally along the device. The stopper may be fixed to a proximal end of the handle actuator rails and disposed along a predetermined longitudinal position of the device between the puller member and the pusher member. The stopper may be configured as a stop against both the puller member being translated proximally and the pusher member being translated distally, beyond the predetermined position. The pusher member may further comprise one or more rails extending distally and longitudinally along the device. The pusher member rails may be configured to slide along the handle actuator rails. The puller member may further comprise channels. The channels may be configured to guide the puller member when the puller member is slid proximally along the handle actuator rails. A distal tip of the inner member may extend distally from the distal end of the outer sheath. The delivery device may include the distal tip with a conductive area configured to cut through a tissue wall. Electrical energy may be supplied to the distal tip through a plug disposed on the pusher member. Radiofrequency energy may be supplied to the distal tip through an electrical plug on the pusher member. A knob may be fixedly attached to the handle actuator.

The above summary of exemplary embodiments, aspects, and/or examples is not intended to describe each embodiment, aspect or example, or every implementation, of the present disclosure. These and other features and advantages of the present disclosure will be readily apparent from the following detailed description, the scope of the claimed invention being set out in the appended claims.

### BRIEF DESCRIPTION OF THE DRAWINGS

The disclosure may be more completely understood in consideration of the following detailed description of various embodiments in connection with the accompanying drawings, in which:

FIG. 1 illustrates a prior art delivery device.

FIG. 2 illustrates a perspective view of a handle body and a distal tip of a delivery device in accordance with one or more embodiments of the present disclosure.

FIG. 3 illustrates a cross section of the delivery device of FIG. 2.

FIG. 4 illustrates an exploded view of the delivery device of FIGS. 2-3.

FIG. 5 illustrates a perspective view of the delivery device of FIGS. 2-4.

FIGS. 6A-6F illustrate perspective views of method steps for employing a delivery device, such as the delivery device of FIGS. 2-5, to deploy a stent, in accordance with one or more embodiments of the present disclosure.

FIGS. 7A-7B illustrate perspective views of method steps for employing a delivery device having a rotatable cam, to deploy a stent, in accordance with one or more embodiments of the present disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

This disclosure is now described with reference to exemplary stent delivery devices, systems and methods that may be used in endoscopic medical procedures. The procedures may be to, for example, access a first organ or lumen by penetrating and creating an opening through a tissue wall of that organ or lumen and placing a stent to maintain the opening on a temporary or prolonged basis. Sometimes, access may include penetrating and creating an opening through a tissue wall of a second organ or lumen that has a tissue wall apposing or adjacent to the tissue wall of the first organ. A stent may be placed to span the openings in the walls with the apposed or adjacent walls engaged between retention members at each end of the stent. The access may be for establishing drainage of one organ into another, creating a path for further instrument access, creating an anastomosis between adjacent tissues walls, such as with a gastrojejunostomy procedure, and the like.

However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings. The same or similar reference numbers will be used throughout the drawings to refer to the same or like parts.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that this numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

It will be understood that the dimensions described in association with one or more embodiments are illustrative only, and that other dimensions are contemplated. The materials that can be used for the various components of the delivery device and the various elements thereof disclosed herein may include those commonly associated with medical devices.

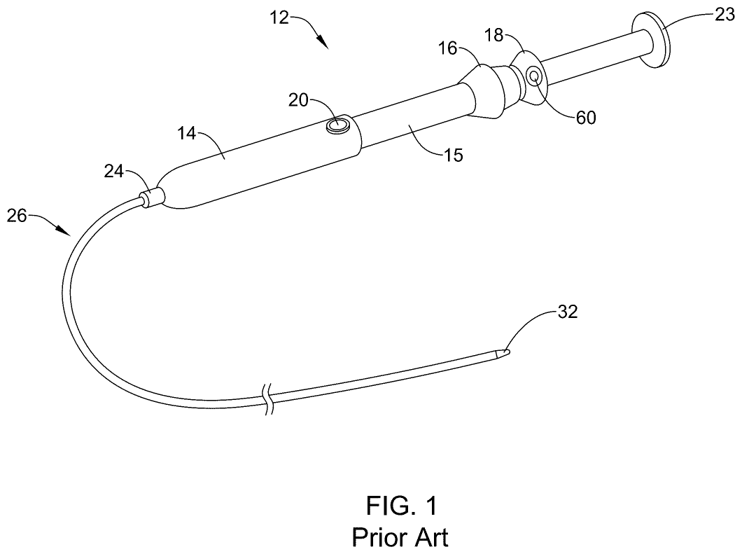

FIG. 1 shows a prior art delivery device, including a device 12, having a handle 14 with a first slide actuator 15, a knob 16, and a lock 20, a second slide actuator 18 with a lock 60, luer lock 24 to secure the handle to the biopsy port of a working channel of an endoscope or other instrument through which the device may be extended, an electrical plug 23, external sheath 26 and a distal tip 32. The distal tip 32 may be attached to an inner shaft and may be slidable through the external sheath 26. The external sheath 26 may be attached to the second slide actuator 18, and be slidable through the handle 14, such that moving the second slide actuator 18 in the proximal direction pulls the external sheath 26 in the proximal direction relative to the inner shaft and the handle. The first slide actuator 15 may be connected to both the external sheath 26 and the inner shaft, such that actuation of the first slide actuator 15 moves the inner shaft and external sheath 26 relative to the handle 14. The lock 20 of the first slide actuator 15 locks the slide actuator 15 relative to the handle 14. Unlocking 20 allows a user to move the first slide actuator 15, moving the inner shaft and external sheath together, effecting how far they extend from the distal end of the handle 14. The lock 60 of the second slide actuator 18 locks the second slide actuator 18 relative to the first slide actuator 15, handle 14 and inner shaft. Unlocking 60 allows the user to pull the second slide actuator 18 proximally relative to the first slide actuator 15, handle 14 and inner shaft, deploying a distal flange of the stent. The second slide actuator may then be fully retracted proximally to deploy the proximal end of the stent.

The distal tip 32 may include a conductive area, configured to cut, heat, and/or cauterize tissue in a patient. The distal tip may be used to create or widen a hole in a tissue wall for the device to pass through prior to deploying the stent. Electrical energy may be supplied to the distal tip 32 in the form of radiofrequency (RF) energy and high frequency (HF) energy. The electrical energy may be supplied from a power source (such as an electrical generator) through the electrical plug 23.

The device may include a self-expanding stent retained for deployment near the distal end of the device. Deployment of the stent may occur upon proximal retraction of the external sheath to deploy a distal flange of the stent, and then proximal retraction of the external sheath to deploy a proximal flange of the stent. This deployment requires two pulling motions by the user, to retract the external sheath proximally, and release the stent from its retained position. Due to the nature of self-expanding stents, stents can be prone to foreshortening. Stents, when constrained, may have a different linear length than when they are fully deployed, further effecting deployment position. This may pose an issue when the delivery location is in a small space, or close to a tissue wall or organ. The anatomy of a desired deployment location may prevent distal movement of the device to compensate for foreshortening of the stent. The two proximal retractions of the external sheath can make it difficult to adjust stent placement during the procedure, and may cause the stent to be placed too far proximally, at the wrong angle, or within the wrong body channel.

A device, according to embodiments of the present disclosure allowing for phased deployment of a stent, including a distal push forward of an inner member of a device while an outer sheath remains stationary to deploy the distal end of the stent, and then a proximal pull backward of the outer sheath while the inner member remains stationary to deploy the proximal end of the stent, may be advantageous and allow for more precise positioning and control over delivery of stents into challenging anatomy, such as small spaces, narrow lumens, spaces requiring a complicated angle of approach, lumens that are typically not-expanded when empty, etc.

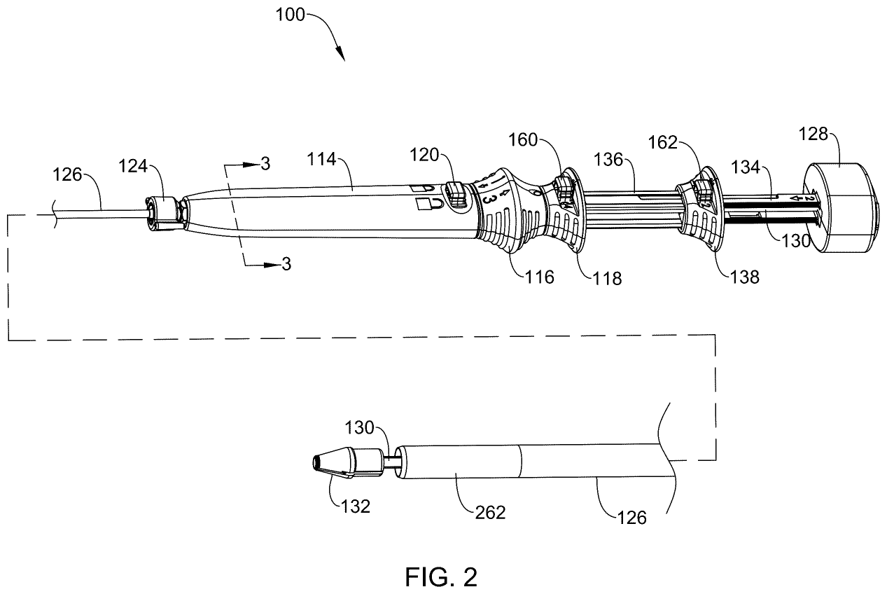

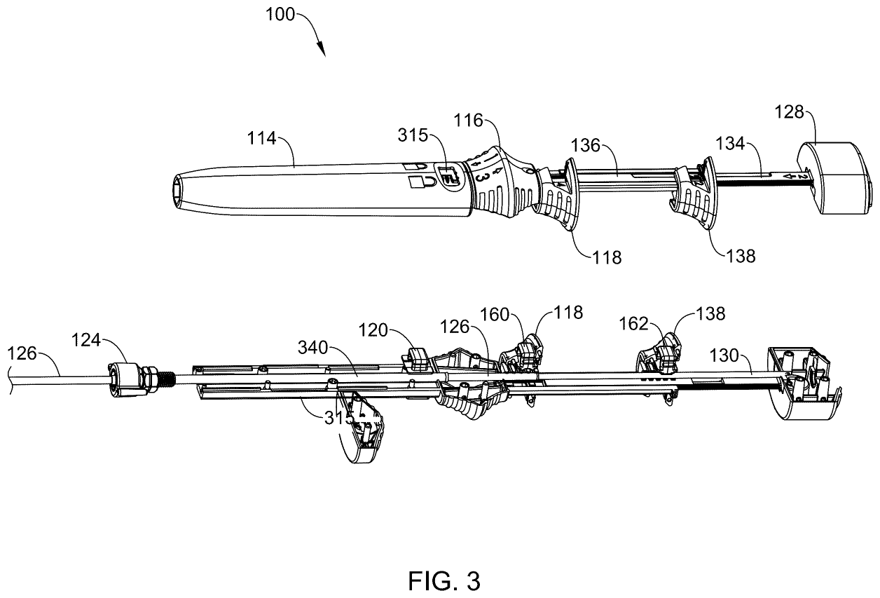

FIG. 2 shows a perspective view of a delivery device 100 according to one or more embodiments of the present disclosure including a pusher member 128, puller member 118, and a stopper 138 located between the pusher member and puller member. In some embodiments, the stopper 138 may be substituted for any type of element that limits movement of the outer sheath 126 and inner member 130, either as a separate element of the device or as element or feature of one of the other components. In some embodiments, there is no stopper 138 between the puller member 118 and pusher member 128. The device also includes a handle housing 114, a scope lock 124 (e.g., luer lock), a knob 116 attached to a handle actuator 315 (FIG. 3), and locks 120, 160, 162, located on the handle housing 114, puller member 118, and stopper 138, respectively. The device includes an outer sheath 126 extending distally out of the distal end of the handle housing 114, and slideably passing through a lumen of the handle housing 114 and scope lock 124.

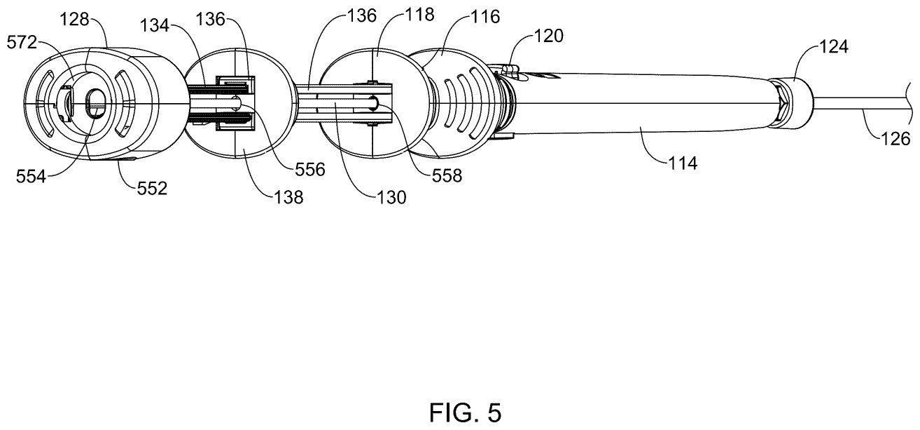

The inner member 130 extends through a lumen of the outer sheath 126 and includes a distal tip 132 on the distal end. The distal tip 132 extends past the distal end of the outer sheath 126. The distal tip 132 and inner member 130 may be a single monolithic element or may be separate elements joined together. In some embodiments, the inner member 130 may include a lumen through which a guidewire, fluid or other means may pass through toward the distal tip 132. In some embodiments, the distal tip may be able to be retracted within the outer sheath. The distal tip 132 may include a conductive area, configured to cut, heat, and/or cauterize tissue in a patient in order to penetrate the tissue and create an opening therethrough. Electrical energy may be supplied to the distal tip in the form of radiofrequency (RF) energy or high frequency (HF) energy. The electrical energy may be supplied from a power source (e.g., electrical generator) to the device through an electrical plug 552 (FIG. 5) on the pusher member.

The pusher member 128 may include a set of upper and lower inner rails 134, slidable along a set of upper and lower outer rails 136 attached to the handle actuator 315 (FIG. 3). The inner rails 134 and pusher member 128 may be a single monolithic element or may be two distinct elements joined together. The pusher member 128 may also include a lock (e.g., luer lock) to allow for attaching a syringe, fluid pump, or the like to the proximal end of the handle. The outer rails 136 and handle actuator 315 may be a single monolithic element or may be two distinct elements joined together. In some embodiments, the inner and outer rails may have a tongue and groove arrangement, ridges, joints, or be interlocking, in order to keep the pusher member 128 in place as it slides longitudinally along the outer rails 136. The inner member 130, is attached to the pusher member 128, such that moving the pusher member 128 and inner rails 134 in the proximal or distal direction along the outer rails of the handle actuator 315 causes movement of the inner member 130 in the proximal or distal direction relative to the handle actuator, handle housing and outer sheath.

The pusher member 128 may be advanced in the distal direction until it reaches the stopper 138. The lock 162 of the stopper 138 may hold the pusher member 128 in place against the stopper 138. The stopper 138 may be fixedly attached to the proximal end of the outer rails 136 of the handle actuator 315 or integral therewith. The stopper 138 may include through channels that the inner rails 134 of the pusher member 128 may pass through as they slide along the outer rails 136 of the handle actuator 315. The through channels of the stopper may be useful to stabilize or guide the inner rails 134 as they slide along the outer rails 136. Movement of the pusher member 128 may extend the inner member 130, and the distal tip 132 relative to and distally out of the outer sheath 126.

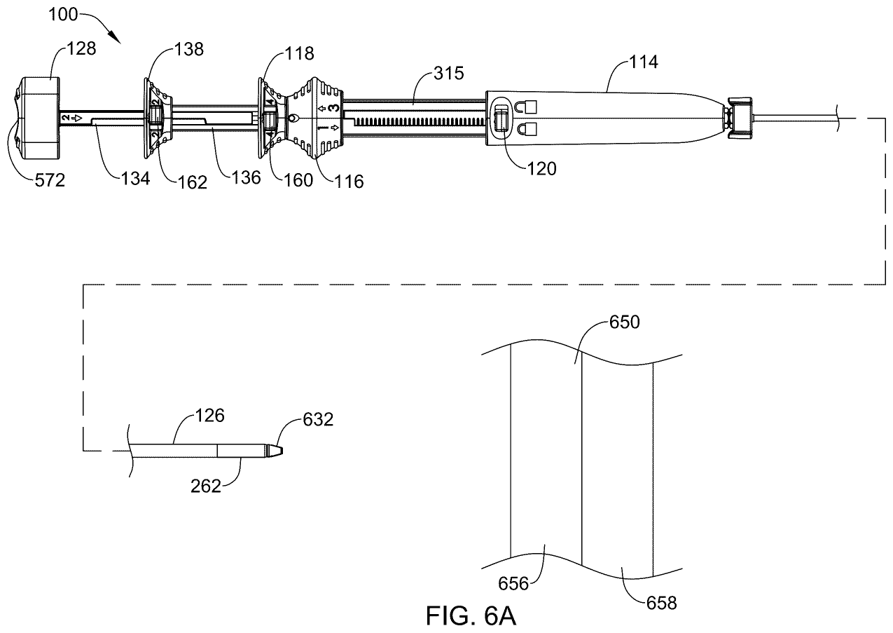

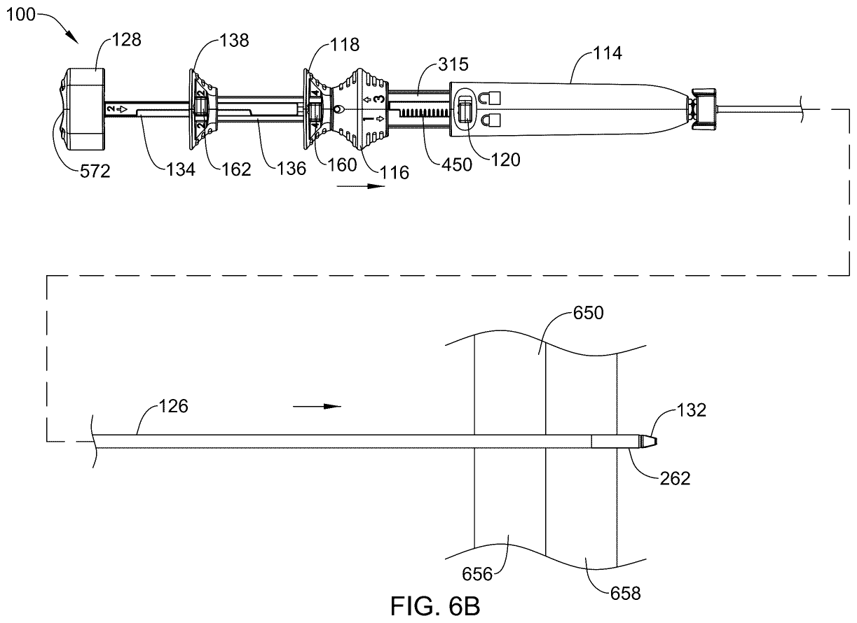

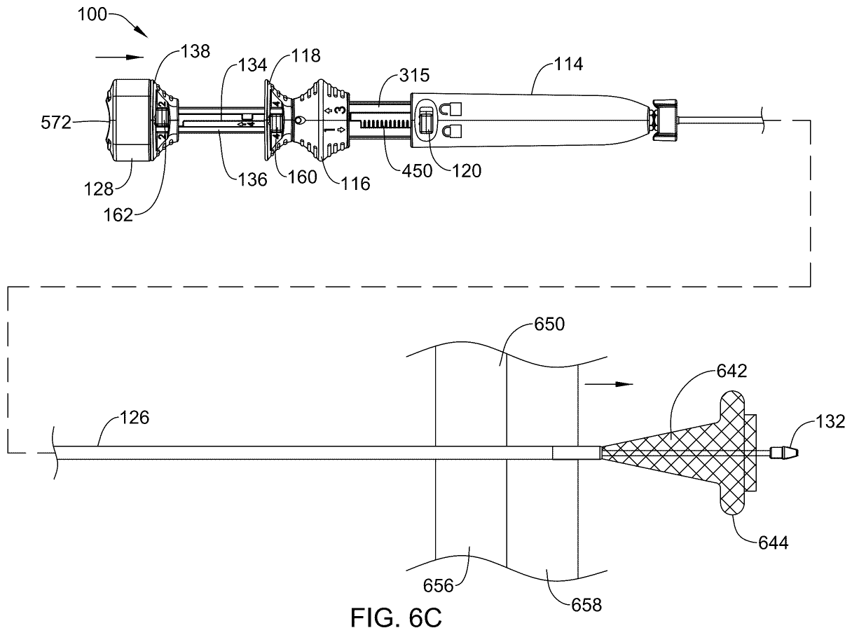

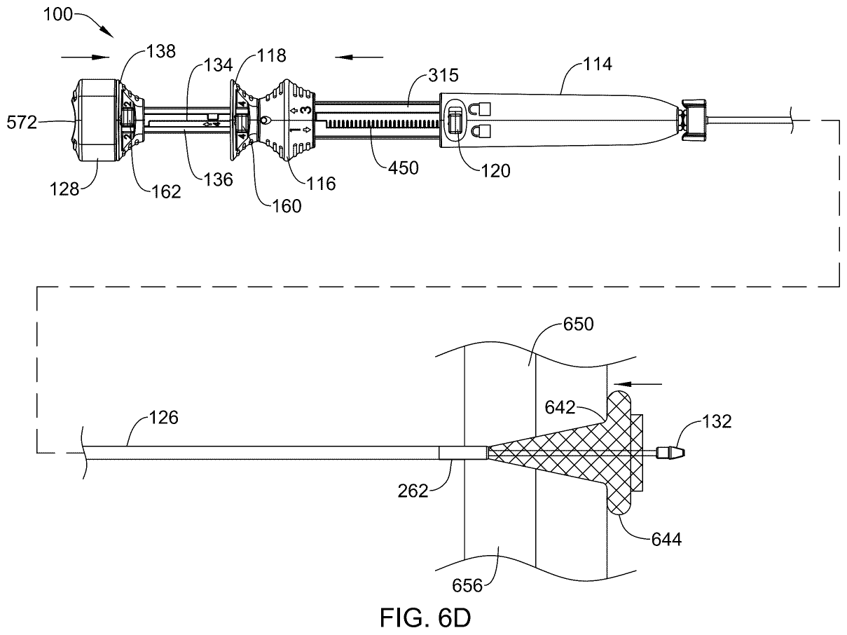

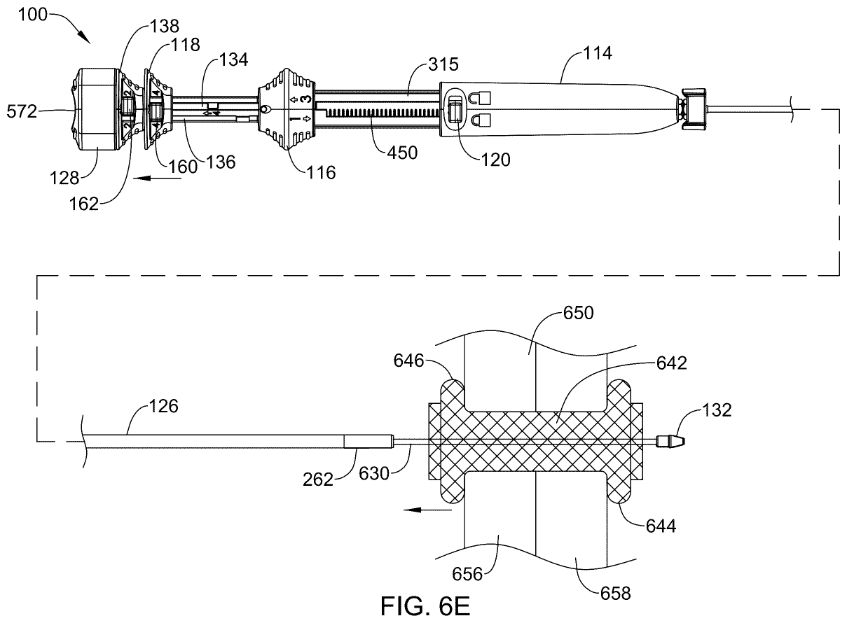

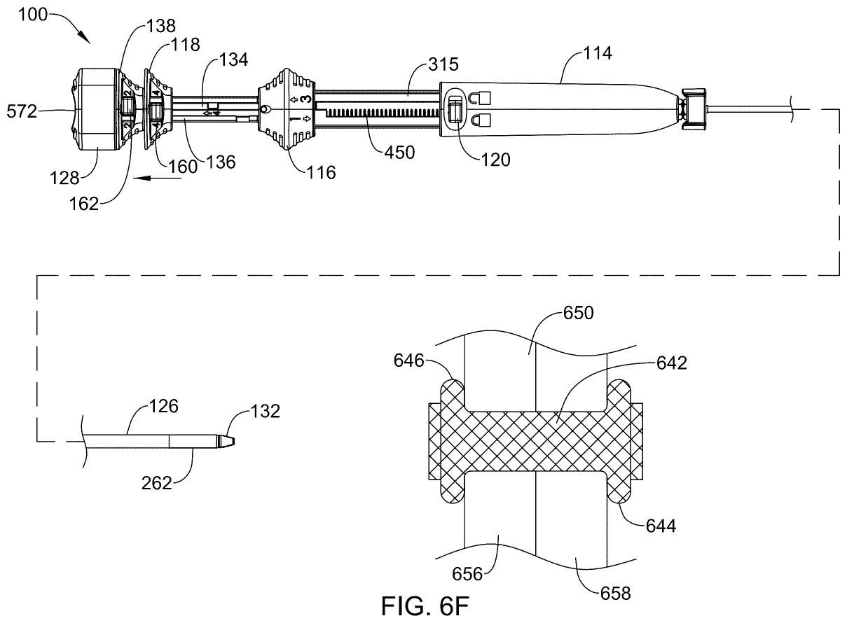

In some embodiments, the inner member 130 and/or outer sheath 126 may have a visible marker 262 that can be visualized after partial retraction of the outer sheath 126. The marker 262 may be obscured by tissue walls before extraction. The position of the marker 262 on the outer sheath and/or inner member may be an indicator that a stent 642 (FIG. 6C) has been deployed a certain amount. The position of the marker 262 may also be an indicator that the outer sheath 126 has been advanced or retracted to a predetermined position. In some embodiments, multiple markers 262 may be used on the inner member 130 and/or outer sheath 126 with various colors or patterns that correspond to various positions of the outer sheath and inner member. The outer sheath and inner member markers may be visible by external imaging, such as x-ray fluoroscopy imaging, directly visible with a scope camera and/or visible by ultrasound.

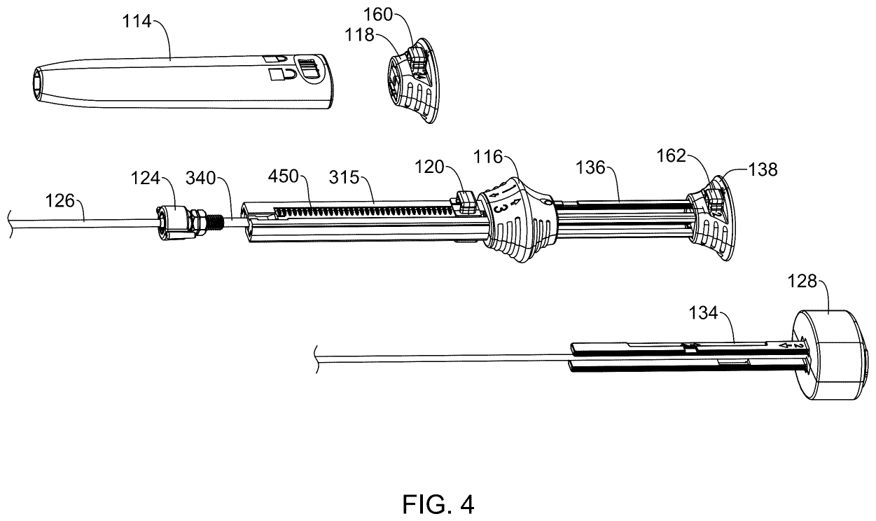

As shown in FIGS. 3 and 4, puller member 118 is slideably coupled to the outer rails 136 of the handle actuator 315. The outer sheath 126 is attached to the puller member 118 such that movement of the puller member 118 in the proximal direction causes movement of the outer sheath 126 in the proximal direction without affecting the position of the inner member 130 and distal tip 132. The handle actuator 315 is coupled to both the inner member 130 and outer sheath 126, with handle body sheath 340 (FIG. 3) surrounding both the inner member 130 and outer sheath 126. The stopper 138 is fixedly attached to the outer rails 136 of the handle actuator 315, stopping the puller member from being displaced past a certain point, which may be a pre-determined position along the device between the puller member and pusher member. The puller member 118 can be pulled proximally until it reaches the stopper 138. The puller member 118 includes a lock 160, that may lock the position of the puller member 118 and outer sheath 130 relative to the handle actuator 315. When unlocked, the puller member is configured to slide along the handle actuator rails pulling the attached external sheath along with it.

When lock 120 is not engaged, the handle actuator 315 may be slid relative to the handle housing 114, moving both the inner member 130 (and lock 162 of stopper engaged) and the outer sheath 126 together. Movement of the handle actuator adjusts the effective working length of the outer sheath 126 and inner member 130, by adjusting how far they extend from the distal end of the handle housing and consequently how far the inner member and outer sheath extend beyond the distal end of a working channel of an instrument (e.g., scope) through which the device may be placed into the body of a patient. The handle actuator 315 includes a knob 116 for a user to grip when pushing or pulling the actuator. The handle actuator 315 also includes a lock 120, that may lock the handle actuator 315 relative to the handle housing 114 and relative to the instrument to which the handle housing is attached with the luer lock 124. The handle actuator 315 includes markings 450 (FIG. 4) to illustrate to a physician how far they have moved the handle actuator relative to the handle housing. The markings 450 may be grooves or may be printed or stamped on. The markings 450 may be raised ribs to interfere with the lock 120 to prevent the handle actuator from moving when the lock 120 is engaged with the ribs. The markings 450 may include an indicator corresponding to a predetermined position of the handle actuator 315 relative to the handle housing 114.

As shown in FIG. 5, the pusher member 128 includes a plug 552. The plug 552 may be configured to deliver electricity to the distal tip 132 of the device in the form of RF or HF energy. The inner member 130 may include a lumen 554. The lumen may extend longitudinally through the length of the inner member 130, such that a guidewire, fluid, or other means may be advanced through the lumen 554 to the distal end of the inner member 130. The pusher member 128 includes a lock 572, e.g., a luer lock or the like. The lock 572 may allow a user to attach a syringe, fluid pump, or the like to the pusher member 128, and flush or inject a fluid, or aspirate a fluid or tissue sample, or the like, through the lumen 554. The syringe or other means attached to the lock 572 may be in fluid communication with the lumen 554 of the inner member 130 allowing the distal end of the inner member 130 to be accessed by a user through the proximal surface of the pusher member.

The stopper 138 includes through-channels aligning the inner rails 134 of the pusher member to the outer rails 136 of the handle actuator. The through-channels are sized and shaped to slideably receive the inner rails 134, allowing the pusher member with inner member attached to slide in the proximal and distal directions. In some embodiments, the inner rails 134 may include notches that prevent the pusher member 128 from being pulled in the proximal direction past a certain point. The notches may prevent the inner rails from being pulled through the stopper 138 in the proximal direction. In some embodiments, the puller member 118 may include through-channels that allow the inner rails to pass through the puller member. The puller member through-channels are sized and shaped to slidably receive the external rails of the handle actuator, such the puller member when pulled proximally slides along and is guided by the external rails of the handle actuator. The stopper 138 further includes a lumen 556 that the inner member 130 can pass through. The puller member may further include a lumen 558 that the inner member can pass through.

FIG. 6A shows an embodiment according to the disclosure of a method of utilizing the device of FIGS. 2-5, with the distal tip 132 positioned next to adjacent body organs or lumens 650 having apposed first tissue wall 656 and second tissue wall 658 (e.g., to access a pseudocyst with second wall for drainage of the cyst through a stent placed in opening through apposed first and second tissue walls into stomach with first tissue wall). In some instances, the body lumen 650 may be a part of the urinary tract, pulmonary tract, gastrointestinal tract, biliary tract, or vasculature. In some embodiments, a stent could be placed across a single wall of a single organ or lumen or across a wall shared by two body organs or lumens. The device of this disclosure may be applied to a variety of procedures in various location within the body. As demonstrated in FIGS. 6A-6F, a stent may be deployed to create a channel or opening between two tissue walls. Stent design and placement may be adjusted to accommodate the unique anatomy of a patient and/or area of the body. As shown in FIG. 6A, the pusher member 128 is extended to its most proximal position, the puller member is in its most distal position, and the handle actuator 315 is fully extended out of the handle housing 114. The distal tip 132 is extending out from the distal end of the outer sheath 126, with the inner member 130 still fully within the outer sheath 126. The locks 120, 160, 162 are engaged to hold the pusher member, puller member, and handle actuator in place. The locks may be released when it is desirable to adjust the position of any of the elements of the device as discussed in more detail below. A guidewire or needle may be advanced through a lumen of the inner member 130 and through the tissue walls prior to passing the distal tip 132 through the walls.

In FIG. 6B, the lock 120 is unlocked, so as to allow the handle actuator 315 to be moved distally. The inner member 130, outer sheath 126 and distal tip 132 move together with the handle actuator, the same distance and direction as the handle actuator 315, because the pusher member and inner member are locked against movement relative to the handle actuator by the engaged stopper lock 162 and the puller member and outer sheath are locked against movement relative to the handle actuator by the engaged lock 160 on the puller member. The distal tip 132 is energized and passed through tissue walls 656, 658. The distal tip 132 may be used to cut, cauterize, or ablate portions of the tissue walls to create an opening in through the tissue walls to allow for easier passage of the device. The distal tip 132 may configured with a smooth, blunt end so as present an atraumatic surface, reducing the potential of causing damage to the tissue walls. The distal tip may be configured to widen a preexisting opening in the tissue wall(s) or create a new opening through the tissue wall(s). A variety of distal tip 132 designs may be used with various embodiments of the device of this disclosure to access, cut, ablate, or cauterize tissue walls.

After moving the handle actuator 315, the lock 120 may be engaged to stop movement of the handle actuator 315 relative to the handle housing 114. With lock 120 engaged and stopper lock 162 disengaged, FIG. 6C shows the pusher member 128 being moved distally until reaching the stopper 138. The distal tip 132 and inner member 130 attached to the pusher member have been moved the same direction and distance as the pusher member 128. As the inner member 130 extends past the end of the outer sheath 126, a distal end of the stent 642 constrained on the inner member 130 by outer sheath 126 is partially exposed and deployed to an unconstrained configuration. The stent 642 includes a distal retention member 644 on the distal end of the stent which forms when the stent is partially exposed. The predetermined position of the stopper along the device, the stent on the inner member, and length of the rails 134 and 136 may be calibrated to allow only the distal retention member 644 to be released when the pusher member 128 is moved to the stopper 138. The length of the rails 134 and 136 may be calibrated to accommodate a variety of different stent shapes and sizes. For example, deployment of a stent with an increased length may require longer rails 134 and 136 to fully deploy the proximal and distal flanges 644, 646.

After the stent 642 is released from its constraint within the outer sheath 126 by distal movement of the inner member relative to the stationary outer sheath, the user may pause to verify the expansion of the distal retention member 644. In some embodiments, ultrasound imaging may be used to visualize the stent 642 position and expansion of retention member 644. In some embodiments, direct visualization of the stent 642 and its expansion may be possible. The stopper lock 162 may then be engaged to prevent further movement of the pusher member and inner member relative to the stopper.

In FIG. 6D, with stopper lock 162 and puller member lock 160 engaged, the lock 120 of handle actuator 315 is unlocked and handle actuator is pulled proximally out of the handle housing 114. Retraction of the handle actuator 315 corresponds to movement of both the outer sheath 126 and inner member 130 relative to the handle body and instrument (e.g., endoscope) to which the handle body is attached via luer lock 124. The distal retention member 644 of the stent 642 keeps the stent from being pulled through the tissue wall(s) as the device is withdrawn. The partially deployed stent may be pulled backward along with the outer sheath 126 and inner member 130, until the distal retention member 644 is pulled snuggly up against the second tissue wall. The lock 120 may then be re-engaged to prevent movement of the handle actuator 315, inner member 130, and outer sheath 126 relative to the handle housing 114. The lock 120 of the handle actuator 315 may be configured to stop movement of the handle actuator 315 proximally beyond a predetermined position (e.g., to prevent handle actuator from being pull proximally completely out of handle body 114). Markers along the external sheath may aid in confirming that the handle actuator has been pulled back proximally a sufficient distance proximal to the first tissue wall to allow the proximal end and proximal retention member of the stent to be properly deployed in the lumen or organ with the first tissue wall.

As shown in FIG. 6E, once the handle actuator has been pulled back to the proper location, lock 120 is reengaged and puller member lock 160 is disengaged, allowing puller member 118 to be pulled proximally along with attached outer sheath 126, and relative to stationary inner member 130. The proximal end of the stent 642 may then be deployed when the outer sheath 126 is pulled back by the user pulling back on the puller member 118. The puller member 118 is pulled proximally until it reaches the stopper 138. The lock 160 may be configured to automatically engage during proximal retraction when the outer sheath is retracted to a predetermined position, or may only be engaged manually. In some embodiments, the puller member 118 may only be locked by lock 160 in the distalmost position of the puller member 118 when the stent is completely undeployed. In other embodiments, the puller member 118 may be locked at any point during proximal retraction by the lock 160. In some embodiments, the puller member 118 may be locked against the stopper 138 to prevent further movement of the outer sheath 126 relative to the inner member 130. In some embodiments, markers 262 on the outer sheath 126 and/or inner member 130 may be visible when the outer sheath 126, or the device together with the outer sheath 162, has been retracted enough to deploy the proximal retention member 646 of the stent 642. The direct visibility of the marker 262 may signal to the user that the stent has been fully deployed, or that the tissue has been sufficiently traversed by the proximal retention member. In other words, direct visibility of the marker 262 may signal to the user that the device and outer sheath have been retracted a sufficient distance proximal to the first tissue wall to allow the proximal end and proximal retention member of the stent to be properly deployed in the lumen or organ with the first tissue wall. Proper placement and/or deployment may be confirmed by fluoroscopy imaging as well. The proximal retention member 646, along with the distal retention member 644 may prevent migration of the stent 642 through tissue walls, 656, 658 after deployment and, when spanning apposed walls, may assist in maintaining the walls in apposition.

As shown in FIG. 6F, when the stent is in its fully deployed configuration, it may be sized and shaped such that the distal tip 132, inner member 130, and outer sheath 126 may be withdrawn from the body lumen, leaving the stent 642 in place.

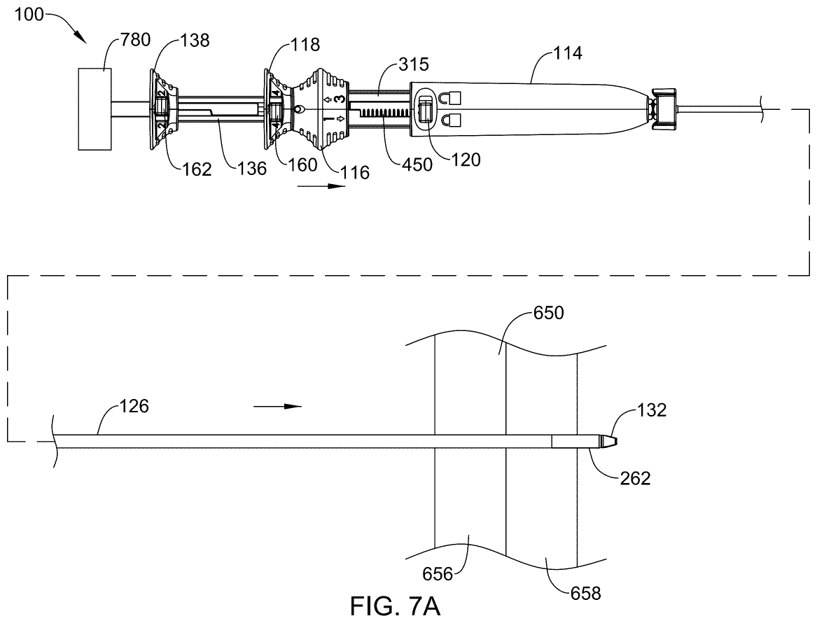

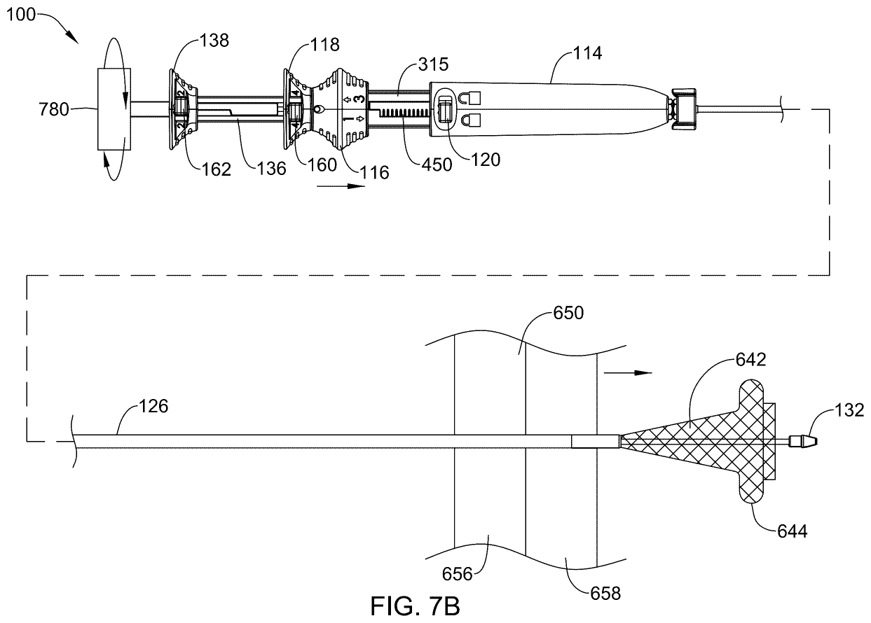

FIG. 7A shows a perspective view of a delivery device 100 according to one or more embodiments of the present disclosure, similar to the embodiment of the device depicted and described with respect to FIG. 6B, except wherein the pusher member 128 is a rotatable cam 780. Though not shown, it should be understood that deployment of a stent with the delivery device of FIG. 7A is essentially the same as deployment of a stent with the delivery device of FIG. 6A-6F. In FIG. 7A, the lock 120 is unlocked, so as to allow the handle actuator 315 to be moved distally. The inner member 130, outer sheath 126 and distal tip 132 move together with the handle actuator, the same distance and direction as the handle actuator 315, because the puller member 118 and outer sheath 126 are locked against movement relative to the handle actuator by the engaged lock 160 on the puller member 118. The distal tip 132 is energized and passed through tissue walls 656, 658. The distal tip 132 may be used to cut, cauterize, or ablate portions of the tissue walls to create an opening through the tissue walls to allow for easier passage of the device. The distal tip 132 may be configured with a smooth, blunt end so as to present an atraumatic surface, reducing the potential of causing damage to the tissue walls. The distal tip may be configured to widen a preexisting opening in the tissue wall(s) or create a new opening through the tissue wall(s). A variety of distal tip 132 designs may be used with various embodiments of the device of this disclosure to access, cut, ablate, or cauterize tissue walls.

After moving the handle actuator 315, the lock 120 may be engaged to stop movement of the handle actuator 315 relative to the handle housing 114. With lock 120 engaged, FIG. 7B shows the cam 780 being rotated. The distal tip 132 and inner member 130 attached to the pusher member 128 are moved in the distal direction in response to rotation of the cam 780. Said differently, rotational movement of the cam 780 corresponds to translational movement of the inner member 130 along the longitudinal axis of the device. As the inner member 130 extends past the end of the outer sheath 126, a distal end of the stent 642 constrained on the inner member 130 by outer sheath 126 is partially exposed and deployed to an unconstrained configuration. The stent 642 includes a distal retention member 644 on the distal end of the stent which forms when the stent is partially exposed. The number of turns that the cam 780 is allowed to rotate may be calibrated to allow only the distal retention member 644 of the stent to be released when the pusher member 128 is moved to the stopper 138. The number of turns that the cam 780 is allowed to rotate may be calibrated to accommodate a variety of different stent shapes and sizes.

After the stent 642 is released from its constraint within the outer sheath 126 by distal movement of the inner member relative to the stationary outer sheath, the user may pause to verify the expansion of the distal retention member 644. In some embodiments, ultrasound imaging may be used to visualize the stent 642 position and expansion of retention member 644. In some embodiments, direct visualization of the stent 642 and its expansion may be possible. In some embodiments, the lock 162 may be configured to lock the cam 780 in place relative to the stopper 138, to prevent further movement of the inner member 130 relative to the delivery device 100. In some embodiments, the cam 780 may include a stopper or lock that can be engaged at any time to prevent rotational movement of the cam 780 and translational movement of the inner member 130. The remaining steps to fully deploy stent 642, including deployment of a proximal retention member 646, may be as described above with respect to deployment of the embodiment of the device with respect to FIGS. 6A-6F.

In some embodiments, the stent may be a self-expanding stent made from a metal, metal alloy, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In at least some embodiments, portions or all of the stent delivery device (and variations, systems or components thereof disclosed herein) may also be doped with, made of, or otherwise include a radiopaque material. The stent itself, delivery device, or both may include radiopaque markers delineating the ends of the stent for placement or the position of the outer sheath with respect to the inner member The radiopaque markers may also delineate the position of the inner member and/or outer sheath with respect to one or more reference points in the body. Markers such as textured or patterned sections that show up on ultrasound or markers that are directly visible may be utilized as well in various combinations and for different or combined purposes. Radiopaque materials are understood to be materials capable of producing a relatively bright image on a fluoroscopy screen or another imaging technique during a medical procedure. This relatively bright image aids a user in determining the location of the stent delivery device (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stent delivery device (and variations, systems or components thereof disclosed herein) to achieve the same result.

In some embodiments, the stent delivery device (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in features and details, particularly in matters of materials, shape, size, and arrangement of steps without exceeding the scope of the disclosure. This may include, to the extent that it is appropriate, the use of any of the features of one example embodiment being used in other embodiments. The scope of the disclosure is, defined by the language in which the appended claims are expressed.

What is claimed is:

1. A delivery device, the device comprising:
an outer sheath having a proximal end, a distal end, and a lumen extending longitudinally therethrough;
an inner member, slideably disposed within the lumen of the outer sheath, the inner member having a distal tip disposed on a distal end of the inner member;
a handle housing having a proximal end, a distal end, and a lumen extending longitudinally therethrough;
a handle actuator slideably disposed within the lumen of the handle housing;
a puller member attached to the proximal end of the outer sheath, the puller member and outer sheath slideably associated with the handle actuator; and
a pusher member attached to the proximal end of the inner member, the pusher member and inner member slideably associated with the handle actuator and the outer sheath;
wherein the outer sheath and inner member are fixable against sliding with respect to the handle actuator, such that translation of the handle actuator relative to the handle housing adjusts the effective working length of the outer sheath and inner member distal to the handle housing;
wherein translation of the puller member slides the outer sheath proximally within the handle actuator and from about the inner member; and
wherein translation of the pusher member slides the inner member distally from within the outer sheath.

2. The device of claim 1, further comprising a stopper having a lock, the stopper fixed in position relative to the handle actuator and disposed along the device between the puller member and the pusher member, the lock when engaged is configured to hold the pusher member in place from sliding relative to the stopper.

3. The device of claim 1, wherein handle housing further comprises a lock, the lock when engaged is configured to hold the handle actuator in place from sliding relative to the handle housing.

4. The device of claim 1, wherein the handle actuator further comprises a lock, the lock when engaged is configured to hold the puller member in place from sliding relative to the handle actuator.

5. The device of claim 1, wherein the handle actuator further comprises one or more rails extending proximally and longitudinally along the device.

6. The device of claim 5, wherein the device further comprises a stopper, the stopper fixed to a proximal end of the handle actuator rails and disposed along a predetermined longitudinal position of the device between the puller member and the pusher member, the stopper configured as a stop against both the puller member being translated proximally and the pusher member being translated distally, beyond the predetermined position.

7. The device of claim 6, wherein the pusher member further comprises one or more rails extending distally and longitudinally along the device, the pusher member rails configured to slide along the handle actuator rails.

8. The device of claim 7, wherein the puller member further comprises channels, the channels configured to guide the puller member when the puller member is slid proximally along the handle actuator rails.

9. The device of claim 1, further comprising a stent constrained for delivery within the lumen of the outer sheath between the outer sheath and the inner member.

10. The device of claim 9, wherein when the inner member is moved distally by a user pushing on the pusher member, a distal end of the stent is deployed, and when the outer sheath is moved proximally by the user pulling on the puller member, a proximal end of the stent is deployed.

11. The device of claim 10, wherein when the stent is fully deployed, it is shaped and sized such that the distal tip of the inner member can be retracted proximally through the stent.

12. A system, the system comprising:
a delivery device, comprising:
an outer sheath having a proximal end, a distal end, and a lumen extending longitudinally therethrough;
an inner member, slideably disposed within the lumen of the outer sheath, the inner member having a distal tip disposed on a distal end of the inner member;
a handle housing having a proximal end, a distal end, and a lumen extending longitudinally therethrough;
a handle actuator slideably disposed within the lumen of the handle housing;
a puller member attached to the proximal end of the outer sheath, the puller member and outer sheath slideably associated with the handle actuator; and
a pusher member attached to the proximal end of the inner member, the pusher member and inner member slideably associated with the handle actuator and the outer sheath;
wherein the outer sheath and inner member are fixable against sliding with respect to the handle actuator, such that translation of the handle actuator relative to the handle housing adjusts the effective working length of the outer sheath and inner member distal to the handle housing;
wherein translation of the puller member slides the outer sheath proximally within the handle actuator and from about the inner member;
wherein translation of the pusher member slides the inner member distally from within the outer sheath; and
a self-expanding stent constrained on the inner member between the inner member and the outer sheath and deployable therefrom by first pushing the inner member with the pusher member relative to the outer sheath and then pulling the outer sheath with the pulling member relative to the inner member.

13. The system of claim 12, wherein when the stent is fully deployed, it is shaped and sized such that the distal tip of the inner member can be retracted proximally through the stent.

14. The system of claim 12, wherein the handle actuator further comprises one or more rails extending proximally and longitudinally along the device.

15. The system of claim 14, wherein the handle housing further comprises a stopper, the stopper fixed to a proximal end of the handle actuator rails and disposed along a predetermined longitudinal position of the device between the puller member and the pusher member, the stopper configured as a stop against both the puller member being translated proximally and the pusher member being translated distally, beyond the predetermined position.

16. The system of claim 14, wherein the pusher member further comprises one or more rails extending distally and longitudinally along the device, the pusher member rails configured to slide along the handle actuator rails.

17. The system of claim 14, wherein the puller member further comprises channels, the channels configured to guide the puller member when the puller member is slid proximally along the handle actuator rails.

18. A method for treatment, the method comprising:
positioning a delivery device carrying a stent in a body of a patient proximate adjacent first and second tissue walls within the body, the delivery device comprising:
an outer sheath,
an inner member slidable within a lumen of the outer sheath, and
a handle body, the handle body comprising:
a handle actuator slidable through a lumen of the handle body;
a puller member attached to the outer sheath and slidable relative to the handle actuator; and
a pusher member attached to the inner member and slidable relative to the handle actuator;
actuating the handle actuator, causing distal translation of the outer sheath and inner member through the first tissue wall and second tissue wall;
pushing the pusher member and inner member distally relative to the handle body and outer sheath, causing deployment of the inner member and a distal end of the stent from within the outer sheath;
translating the inner member and outer sheath together proximally relative to the handle body to engage the deployed distal end of the stent against the second tissue wall; and
pulling the puller member and outer sheath proximally relative to the handle body and inner member, causing deployment of the proximal end of the stent against the first tissue wall and securing the first and second tissue walls between the distal and proximal stent ends.

19. The method of claim 18, the device further comprising a stopper having distal and proximal surfaces fixed in position relative to the handle actuator between the pusher member and puller member.

20. The method of claim 19, wherein distal movement of the pusher member and inner member during the pushing step is limited by the pusher member butting up against the proximal surface of the stopper and proximal movement of the pulling member and outer sheath during the pulling step is limited by the puller member butting up against the distal surface of the stopper.
